(19) United States
(12) Patent Application Publication
(10) Pub. No.: US 2005/0035610 A1
(43) Pub. Date: Feb. 17, 2005

(54) Contact lens insertion and removal device

(76) Inventor: Jerry Butler, Shreveport, LA (US)

Correspondence Address: Ronald B. Sefrna;Sefrna & Associates, P.O. Box 567, Tyler, TX, 75710, US

(21) Appl. No.: 10/913,043

(22) Filed: Aug. 6, 2004

Related U.S. Application Data
(60) Provisional application No. 60/494,171, filed on Aug. 11, 2003.

(51) Int. Cl. A61F 9/00

(52) U.S. Cl. 294/1.2

(57) ABSTRACT

A contact lens insertion and removal device includes a lens cup with a concave curvature for engaging a contact lens and a central aperture, a deformable shape retentive handle with a limited interior volume from which air can be exchanged between the interior and exterior of the handle, and an elongate hollow wand connected between the handle and the lens cup to form a passageway from the interior of the handle through the central aperture of the lens cup. A contact lens may be engaged with limited suction by the lens cup and released from the lens cup by manipulation of the handle without risk of damage to the contact lens.

### RELATED APPLICATION DATA

This application claims the benefits of U.S. Provisional Patent Application Ser. No. 60/494,171, filed Aug. 8, 2003, titled “Contact Lens Insertion And Removal Device”.

### FIELD OF THE INVENTION

The present invention generally relates to the field of instruments for handling contact lenses, and in its preferred embodiments more specifically relates to an instrument or device for inserting and removing contact lenses, including soft contact lenses, from the eye.

### BACKGROUND

Since the advent of contact lenses users of the lenses have experienced difficulty inserting and/or removing the lenses. Typically, the tip of a finger is used to insert the lenses and assist in removing them, but that procedure often has disadvantages. Removal of contact lenses can be particularly problematic, because the placement of a finger near the eye to remove or to catch a lens obscures visibility with the other eye and makes it difficult, if not impossible, to see and locate the lens to be removed.

Various approaches to solving these problems have been tried, but none have proven to be fully successful. Some approaches known in the prior art are based upon the use of fairly complex devices that include components for, for example, holding open the eyelids as well as components for holding and inserting a contact lens. These devices are subject to breakage and are often too bulky for a contact lens wearer to easily carry along for use when the need to remove or insert a lens may arise.

Another approach uses a suction or vacuum to hold a contact lens at the end of a tool for insertion into the eye. With some devices of this type it is difficult to release the vacuum to release the lens once it is in place on the eye. With other devices of this general type it is difficult to control the suction, and an excessive suction may be imposed, damaging the lens. One device known in the prior art attempts to address that problem by providing a barrier to prevent sucking the lens into the suction tube of the device, but does not address the problem of excessive suction directly.

### SUMMARY OF THE INVENTION

The present invention provides a device for inserting and removing contact lenses from the eye of a user that overcomes the problems and disadvantages of the prior art. The device of the invention is effective and easy to use, and prevents excessive suction and the associated risk of damage to the contact lens and/or to the eye by limiting the vacuum that can be created by the device.

The device of the invention includes the basic components of a lens cup, a handle, and a hollow wand connected between the lens cup and the handle. The lens cup is formed as a curved disk with a central aperture through which air can move during use of the device. The handle is formed of a shape retentive material that can be compressed or deformed by squeezing, and includes in its interior a controlled volume of air space. As the handle is squeezed a limited volume of air is expelled, and when the handle is released a limited volume of air is drawn into the interior of the handle as it returns to its original configuration.

In the device of the invention, the wand forms an air passageway between the lens cup and the handle, so that the volume of air moved by deformation and return of the handle is directed through the wand and the central aperture of the lens cup. The curvature of the lens cup generally matches the curvature of a contact lens and the lens cup is of smaller diameter than a contact lens, so that the lens cup can function as a suction cup to engage a contact lens.

To insert a lens, a user squeezes the handle to expel the limited volume of air from the handle interior, places the lens cup against a contact lens, and releases the handle to create a sufficient suction to hold the contact lens against the lens cup without damaging or drawing the contact lens too far into the lens cup. The device is then used to move the contact lens to and onto the user's eye in the desired position. The user then again squeezed the handle to expel air, breaking the suction and releasing the contact lens. To remove a contact lens removal the user simply reverses the steps for insertion.

The limited air space in the handle that can be moved in and out of the handle regardless of how hard a user squeezes inherently limits the suction that can be imposed upon a contact lens, eliminating the risk of damage to the contact lens.

The structure and features of the device of the invention will be described in more detail with reference to the accompanying drawing figures.

### BRIEF DESCRIPTION OF THE DRAWING FIGURES

FIG. 1 is a perspective view of the preferred embodiment of the device of the invention.

FIG. 2 is a side view of the preferred embodiment of the device of the invention, with the components in separated relation FIG. 3 is a sectioned side view of the handle component of the preferred embodiment of the device of the invention.

FIG. 4 is a sectioned side view of an alternative embodiment of the handle component.

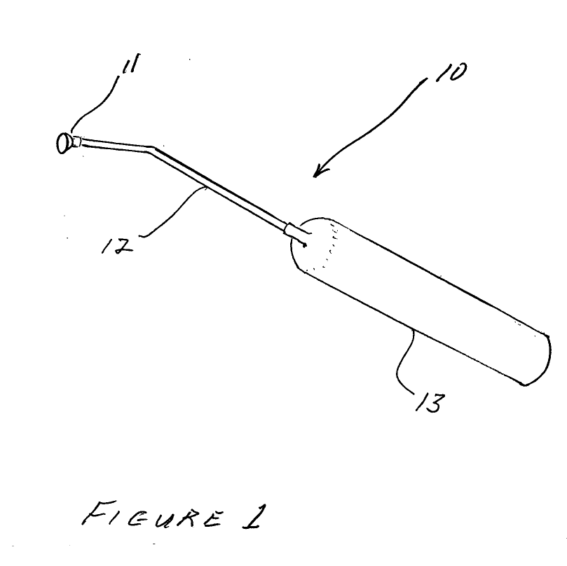

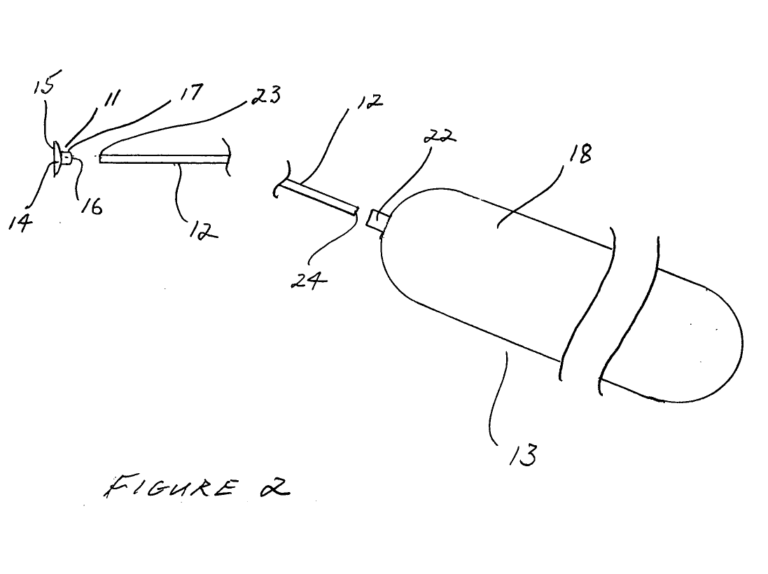

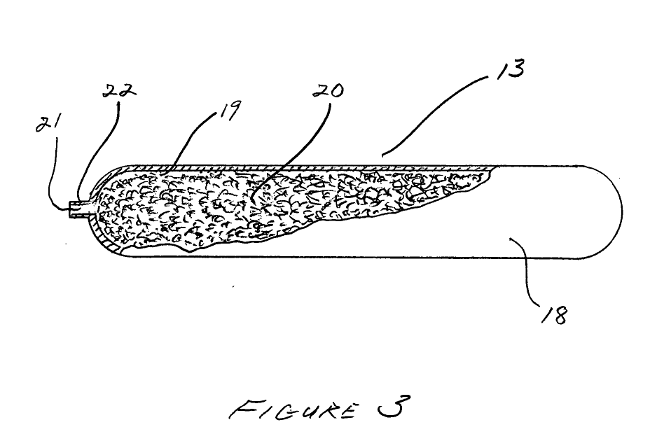

### DESCRIPTION OF THE INVENTION

Referring to the drawing figures, the device of the invention, generally designated by reference numeral 10, comprises the primary components of a lens cup 11, an elongate wand 12, and a handle 13.

Lens cup 11 is shaped as a suction cup, with a thin, flexible wall 14 of a non-abrasive material, formed in a curved configuration with a radius of curvature slightly less than the typical radius of curvature of a contact lens. In the preferred embodiment the lens cup is configured as a section of the wall of a hollow sphere. Wall 14 thus surrounds an open concave interior, and has a circular outer edge 15. The diameter of the lens cup at the outer edge is preferably significantly less than the diameter of a typical contact lens. Wall 14 is penetrated by a suction aperture 16 that is preferably centered in wall 14, with its axis perpendicular to the plane defined by edge 15. Suction aperture 16 is surrounded by an annular lens cup collar 17. The lens cup is preferably constructed of a silicone material, or other material suitable for use in and around the eyes.

Handle 13 is preferably a slightly elongate hollow body with an outer wall 18 formed of a shape retentive, but deformable material. The handle is shaped to be easily grasped and held by a user during the process of inserting or removing a contact lens. The outer surface of wall 18 may be smooth, or may be formed with a textured surface to increase friction and prevent a user's fingers from slipping on the handle surface during use, since the handle and/or the user's fingers may be damp or wet. In the preferred embodiment the interior 19 of the handle is substantially filled with a filler material 20 such as a resilient closed cell foam that occupies the majority of the volume of the interior of the handle, so that only a limited portion of the interior volume of the handle is occupied by air or other gas that is not confined within the structure of the filler material. The outer wall of the handle is penetrated by a wand aperture 21, preferably disposed at one end of the handle in coaxial alignment with the longitudinal axis of the handle. The wand aperture is surrounded by an annular handle collar 22, similar to lens cup collar 17, creating a passageway through the outer wall to the interior of the handle.

Wand 12 is an elongate hollow tube with a first end 23, to be received in lens cup collar 17, and a second end 24, to be received in handle collar 22, connecting the lens cup to the handle and providing a passageway from the cavity of the lens cup to the handle through the interior of the wand.

The device of the invention is used to insert a contact lens by squeezing the handle to expel air from the interior of the handle through the wand. Before the handle is released a contact lens is placed onto the lens cup, or alternatively, the lens cup is placed against a contact lens resting in a holder or on the user's finger or palm. The squeezing force on the handle is then released so the shape retentive material of the outer wall seeks to return to its rest configuration, creating a low vacuum in the lens cup and holding the contact lens against the outer edge of the wall of the cup. With the lens held in place on the cup, the device is used to move the lens to the eye in which the lens is to be inserted, and to place the lens in the correct position against the eye. The surface tension of the ocular fluid will tend to hold the lens against the eye as the lens is released from the lens cup. To release the lens pressure is again applied to the handle, decreasing the volume of the interior of the handle and releasing the vacuum holding the lens cup against the lens, thereby releasing the lens cup from the lens, which remains in the proper position on the eye. The device may then be moved away from the eye.

To remove a contact lens from the eye the installation steps are essentially performed in reverse. The handle of the device is squeezed to expel air and reduce the interior volume of the handle. While the handle is being squeezed, the lens cup is place against a contact lens in the eye, and the pressure on the handle is released, creating a low vacuum to hold the lens against the lens cup. The vacuum is sufficient to hold the contact lens against the lens cup with a force slightly greater than the force associated with the surface tension of the ocular fluid that holds the contact lens against the eye. The device is then moved away from the eye, to remove the contact lens from the eye. The lens is released from the lens cup by again squeezing the handle to release the vacuum.

One of the significant features of the device of the invention is the limitation of the volume of air that can be displaced and moved through the wand as the handle is squeezed and released, and, accordingly, the limitation of the degree of vacuum that can be created by the manipulation of the device. In the embodiment of the device described above, the “free” volume of the interior of the handle is limited because the majority of the volume of the interior of the handle is occupied by, e.g., a resilient closed cell foam. The closed cell foam provides a soft feel to the handle, but the air (or other gas) contained in the cells of the foam remains captured in the cells and does not contribute to the volume of air that can be moved in and out of the handle when it is squeezed and released. Since only a limited volume of air can be expelled from the interior of the handle, regardless of how much squeezing force is applied, the vacuum created at the lens cup when the handle is released can be selected to remain within a safe range that will not harm a contact lens or risk “sucking” the lens too far into the lens cup. Since the vacuum holding the contact lens against the lens cup is limited by the structure of the device, safe use of the device is not dependent upon the exercise of great care by the user, who is then able to concentrate on placing the contact lens in the eye for insertion and on proper placement of the lens cup against the lens for removal.

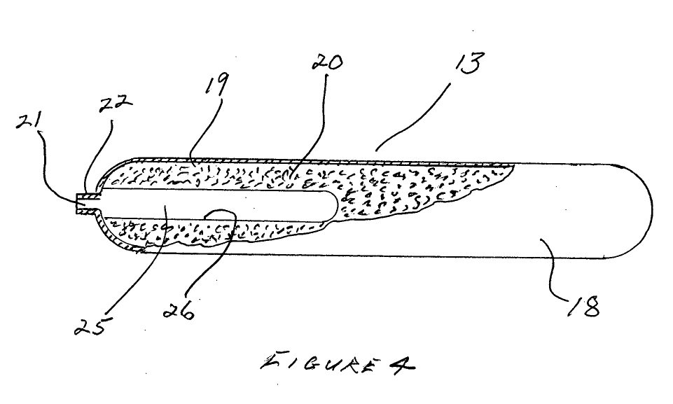

Although the preferred means of limiting the vacuum or suction that can be imposed is the limitation of the free internal volume of the handle, it is contemplated that other means of control may be employed. For example, the interior 19 of the handle may be divided, so that air can be dispelled and returned to only an open section 25 surrounded by an inner wall 26 as shown in FIG. 4, with filler material omitted from that section. Other embodiments and various other means of limiting the volume of air that can be exchanged through the lens cup can be devised, while providing a handle of sufficient size to be easily held and manipulated by a user; all within the scope of the invention.

Other variations can be made to make the device of the invention very accommodating to the preferences of individual users. For example, a small mirror could be connected to the handle to allow a user to more easily view and control the insertion and removal process. The wand could be made of a deformable material, so that it could be bent into different configurations to suit the preferences of individual users without closing the interior passageway. The wand could be made of a material that can be readily cut with, e.g., household scissors, and the wand releaseably connected to the handle collar, so the wand could be removed from the handle and shortened to a length desired by an individual user and then reinserted in sealed relation with the handle collar.

The foregoing description of the preferred embodiment and of certain contemplated variations and alternatives is for purposes of illustration, and is not limiting of the scope of the invention, which is to be broadly construed. It is to be understood that the invention is susceptible to further variations and alternative embodiments, all within the reasonable scope and breadth thereof.

1. A device for inserting and removing a contact lens from an eye, comprising
a lens cup for engaging the outer surface of a contact lens, with a thin flexible curved wall, having a concave inner surface and a convex outer surface, and a suction aperture penetrating said wall;
a flexible shape retentive handle with an outer wall surrounding and enclosing an interior, a handle aperture penetrating said outer wall to form a passageway between the interior of said handle and the exterior of said handle, said interior of said handle having a first volume in air flow communication with said exterior of said handle through said handle aperture, and a second volume closed against air flow communication with said exterior of said handle; and
an elongate hollow open ended wand with a first end and a second end, said first end of said want being interconnected to said outer surface of said lens cup around said suction aperture, and said second end of said wand being interconnected to said handle around said handle aperture, said wand forming an air flow passageway between said first volume of said interior of said handle through said suction aperture of said lens cup.

2. The device of claim 1, wherein said lens cup further comprises a hollow open ended lens cup collar connected to and extending outwardly from said outer surface of said wall around said central aperture, with said first end of said wand received in said collar.

3. The device of claim 1, wherein said handle further comprises a hollow open ended handle collar connected to said outer wall of said handle around said handle aperture, with said second end of said wand received in said handle collar.

4. The device of claim 1, wherein the majority of said interior of said handle is filled with a resilient closed cell foam material to form said second volume of said interior of said handle, and wherein said first volume of said interior of said handle comprises the portion of said interior of said handle not filled with said foam material.

5. The device of claim 1, wherein said first volume of said interior of said handle is substantially smaller than said second volume of said interior of said handle.

6. The device of claim 1, wherein said handle includes an interior wall dividing said interior of said handle into said first volume and said second volume.

7. The device of claim 1, wherein said second volume is filled with a resilient material.

8. The device of claim 6, wherein said first volume is substantially smaller than said second volume.

9. The device of claim 6, wherein said inner wall is formed as a hollow bulb and wherein said inner wall is connected to said outer wall of said handle around said handle aperture such that said passageway formed by said handle aperture is between the interior of said bulb and the exterior of said handle.

10. The device of claim 1, wherein said outer wall of said handle has an outer surface and wherein said outer surface of said outer wall is formed with a slip resistant texture.

11. A method of inserting a contact lens into an eye using an insertion device having a lens cup for engaging the contact lens, a handle with an air space in the interior thereof in air flow communication with the exterior of the handle, the air space being substantially smaller in volume than the volume of the handle, and a hollow wand connected between the lens cup and the handle to form a passageway for the movement of air between the air space in the handle and the lens cup, comprising the steps of
compressing the handle, thereby decreasing the volume of the air space in the handle and expelling a limited volume of air from the air space;
bringing the lens cup into contact with the outer surface of a contact lens such that the contact lens occludes the passageway from the lens cup to the handle;
releasing the compression on the handle, thereby allowing expansion of the air space in the handle, creating a partial vacuum in the passageway and between the lens cup and the contact lens sufficient to hold the contact lens against the lens cap without damaging or significantly deforming the contact lens;
manipulating the handle to bring the contact lens to the eye in which it is to be inserted and into contact with the eye; and
compressing the handle to decrease the volume of the air space in the handle and expel air therefrom, thereby releasing the vacuum in the passageway and releasing the lens cup from the contact lens.

12. A method of removing a contact lens from an eye using an insertion device having a lens cup for engaging the contact lens, a handle with an air space in the interior thereof in air flow communication with the exterior of the handle, the air space being substantially smaller in volume than the volume of the handle, and a hollow wand connected between the lens cup and the handle to form a passageway for the movement of air between the air space in the handle and the lens cup, comprising the steps of
compressing the handle, thereby decreasing the volume of the air space in the handle and expelling a limited volume of air from the air space;
manipulating the handle to bring the lens cup to the eye from which the contact lens is to be removed and into contact with the outer surface of a contact lens such that the contact lens occludes the passageway from the lens cup to the handle;
releasing the compression on the handle, thereby allowing expansion of the air space in the handle, creating a partial vacuum in the passageway and between the lens cup and the contact lens sufficient to hold the contact lens against the lens cap with a force greater than the surface tension force holding the contact lens against the eye but insufficient to damage or significantly deform the contact lens;
manipulating the handle to lift the contact lens from the eye.

13. The method of claim 12, comprising the further step of
compressing the handle to decrease the volume of the air space in the handle and expel air therefrom, thereby releasing the vacuum in the passageway and releasing the lens cup from the contact lens.
